(19) United States
(12) Patent Application Publication
(10) Pub. No.: US 2024/0374468 A1
(43) Pub. Date: Nov. 14, 2024

(54) A RED-LIGHT THERAPY MASSAGE GUN ATTACHMENT

(71) Applicant: Bradley CARDEN, Manly Vale (AU)

(72) Inventor: Bradley CARDEN, Manly Vale (AU)

(21) Appl. No.: 18/293,174

(22) PCT Filed: Jul. 28, 2022
(86) PCT No.: PCT/AU2022/050792

§ 371 Date: Jan. 29, 2024

(30) Foreign Application Priority Data
Aug. 9, 2021 (AU) 2021902464

(51) Int. Cl. A61H 23/00; A61H 23/02

(52) U.S. Cl. CPC A61H 23/006; A61H 23/0254; A61H 2201/0153; A61H 2201/10; A61H 2201/5007

(57) ABSTRACT

A red-light therapy massage gun attachment has a housing defining a massage gun driveshaft engagement at a proximal side of the housing and an impact surface at an opposite distal side of the housing. The housing contains at least one light source element therein operative to emit light in a wavelength range between 600 nm and 1100 nm from the impact surface.

### FIELD OF THE INVENTION

This invention relates generally to an attachment which attaches to a driveshaft of a massage gun for delivering musculoskeletal massage therapy, and which is configured to simultaneously emit red light tissue therapy in the range of 600 nm and 1100 nm.

### SUMMARY OF THE DISCLOSURE

There is provided a red-light therapy massage gun attachment which may be used both for exercise preconditioning or after exercise to improve musculoskeletal recovery and improve sports performance in athletes and the like.

The attachment comprises a housing defining a massage gun driveshaft engagement at a proximal side of the housing and an impact surface at an opposite distal side of the housing. The attachment is attached to the massage gun driveshaft using the engagement in use.

The housing contains at least one light source element therein operative to emit light in a wavelength range between 600 nm and 1100 nm from the impact surface.

As such, the attachment which applies Photobiomodulation (PBM) in the form of red or near-infrared (NIR) light in addition to massage pressure to stimulate, heal, and regenerate damaged tissue.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a massage gun attachment in accordance with an embodiment engaging a massage gun;

FIG. 2 shows a side perspective view of the attachment;

FIG. 3 shows a proximal view of the attachment;

FIG. 4 shows a distal view of the attachment;

FIG. 5 shows the attachment disassembled;

FIG. 6 shows an exemplary circuit diagram of the attachment in accordance with an embodiment;

FIG. 7 shows a side internal view of the attachment;

FIG. 8 shows a side internal view illustrating interconnection of the attachment to the massage gun in accordance with an embodiment;

FIG. 9 shows a side internal view illustrating interconnection of the attachment to the massage gun in accordance with a further embodiment; and

FIG. 10 shows a proximal view of the attachment in accordance with an embodiment.

### DESCRIPTION OF EMBODIMENTS

A red-light therapy massage gun attachment 100 is configured to attach to a massage gun 102. The massage gun 102 comprise a handle 103 concealing a battery supply 104 therein which turns an electric motor 105 (or reciprocating electromagnets or the like) within a head 106 thereof which reciprocates a driveshaft 107.

The attachment 100 comprises a housing 101 defining a massage gun driveshaft engagement 108 at a proximal side of the housing 101 and an impact surface 109 at an opposite distal side of the housing 101.

The housing 101 further contains at least one light source element 110 therein operative to emit light 111 in a wavelength range of between 600 nm and 1100 nm from the impact surface 109.

The housing 101 may define a disc-like head 112 defining the engagement 108 at a proximal side thereof and wherein a distal side of the disc-like head 112 defines the impact surface 109 and is convex. The radius of the convexity of the disc-like head 112 may be much greater than the width of the impact surface 109 thereby conferring a relatively wide and suitably ergonomic convex impact surface 109 despite the small size of the attachment 100.

FIG. 2 shows the attachment 100 disassembled in accordance with an embodiment wherein the attachment 100 may comprise a plastic base 113 defining the proximal portion of the disc-like head 112 and the engagement 108.

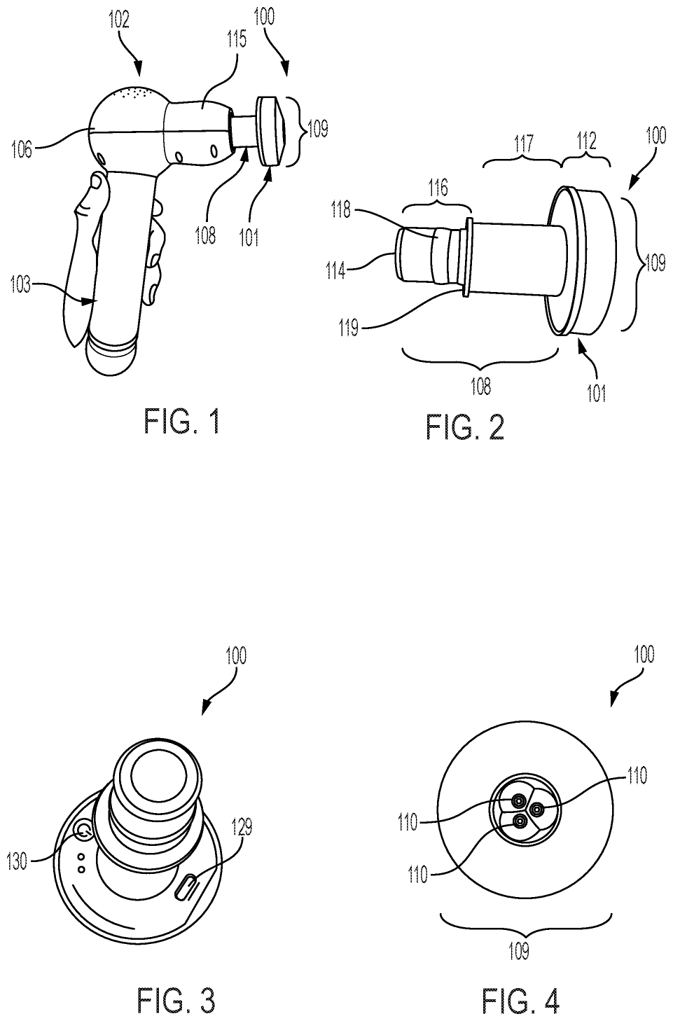

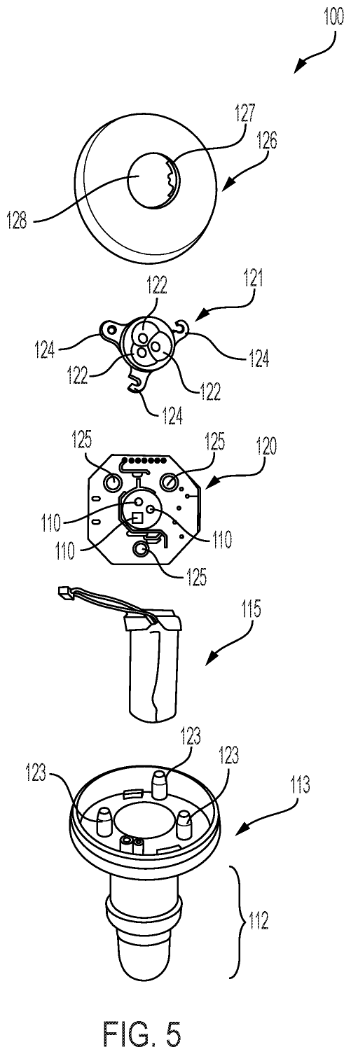

In the embodiment shown, and with further reference to FIG. 2, the engagement 108 may comprise a shaft 114 configured to fit within a barrel 115 of the massage gun 102 in the manner shown in FIG. 1 and which engages the driveshaft 107 of the massage gun 102.

The shaft 114 may be hollow to conceal a battery 115 therein.

As shown in FIG. 2, the shaft 114 may comprise a proximal section 116 and a distal section 117. The proximal section 116 may be narrower than the distal section 117. The proximal section 116 may comprise an interference fit rim 118 which fits within a corresponding recess of the massage gun driveshaft 107. A rubberised impact pad 119 may interface the sections 116, 117.

In an alternative embodiment shown in FIGS. 9 and 10, the engagement 108 comprises a recess 134 and wherein a distal end of the massage gun driveshaft 107 fits in the recess 134.

The housing 101 may further contain a PCB 120 having the light source elements 110 mounted thereon. The light source elements may be PCB mounted LEDs. The planar PCB 120 may fit within the disc-like head 112.

The attachment 100 may further comprise a reflector 121 comprising recessed reflectors 120 for each light source element 110. These recessed reflectors 120 suitably focus or spread the light 111 emitted by the light source elements 110 towards the skin of the user in use.

The plastic base 113 may comprise hollow posts 123 through which screws may secure side engagements 124 of the reflector 121 and through apertures 125 of the PCB 120.

The attachment 100 may further comprise a cover piece 126 which defines the impact surface 109 and which fits to the plastic base 113 to complete the disc-like head 112. The cover piece 126 may define a central window 127 having a transparent cover 128 secured therein which continues the convex curvature of the impact surface 109. In embodiments, the transparent cover 128 may be further shaped to act as a lens to further focus or spread the light emitted by the light source elements 110.

Preferably, at least a portion of the distal side of the attachment 100 comprises reflective material. In this regard, the cover piece 126 may be brushed aluminium to thereby reflect any light reflected from the skin back towards the skin to increase the effective intensity thereof.

As shown in FIG. 3, the attachment 100 may comprise a recharging port 129 for recharging the battery 115. The recharging port 129 may comprise a USB-C connector. As is evident from FIG. 3, the recharging port 129 may be concealed on the proximal side of the disc-like head 112.

As is further shown in FIG. 3, the attachment 100 may comprise a power button 130 to control the operation of the light source elements 110. Similarly, the power button 130 may be concealed on the proximal side of the disc-like head 112. In embodiments, repeated pressing of the power button 130 may step through operational modes, including operational modes wherein the light source elements 110 emit light various wavelength ranges and/or intensities.

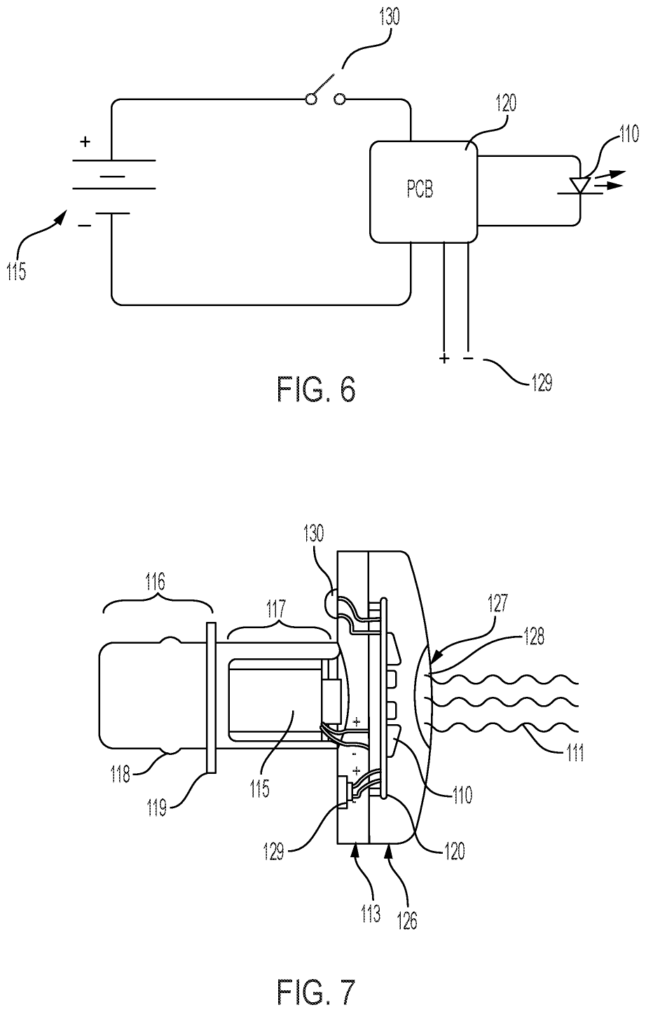

FIG. 6 shows an exemplary electrical schematic wherein the PCB 120 interfaces the battery 115 via the power button 130 to control the light source elements 110 and further interfaces the recharging port 129.

The PCB 120 may contain a microcontroller for controlling the operation of the attachment 100.

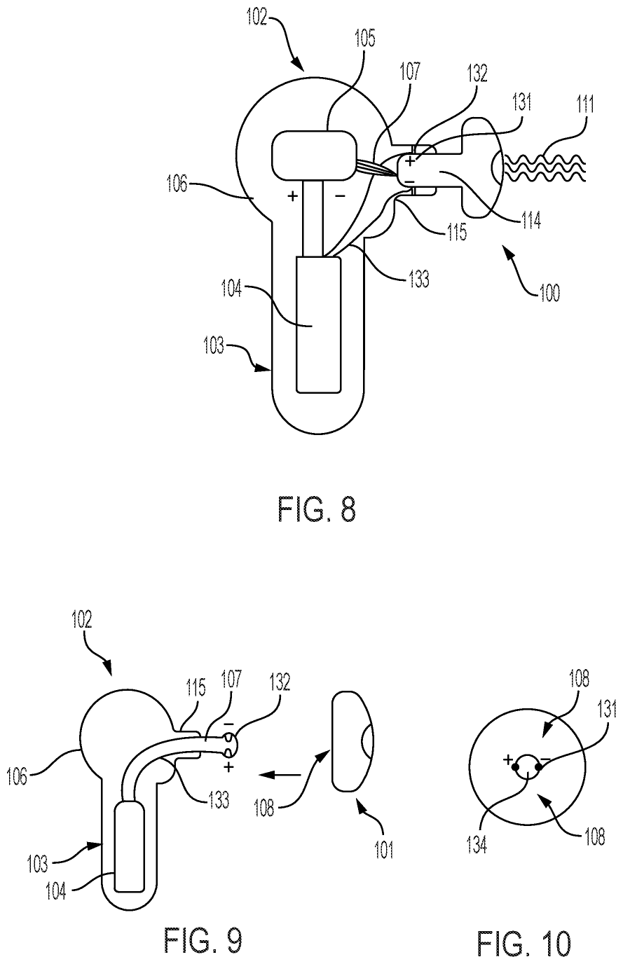

In embodiments, as opposed to the attachment 100 comprising a battery 115, the attachment 100 may draw power directly from the massage gun 120. As shown in FIGS. 8 and 9, the barrel 115 or the driveshaft 107 of the massage gun 120 may expose electrical contacts 132 which draw power from the battery 104 thereof by electrical leads 133. Correspondingly, the attachment 100 may expose electrical contacts 131 which electrically connect the electrical contacts 132 of the massage gun 102 to draw power therefrom.

In further embodiments, attachment 100 comprises the battery 115 which is rechargeable and wherein the electrical contacts 131, 132 are used to recharge the battery 115.

As alluded to above, the attachment 100 may comprise a wavelength selector and wherein the wavelength selector is operable to cause the light source elements 110 to emit light within at least two wavelength ranges. For example, the wavelength selector may be operable to cause the light source elements 110 to emit wavelength ranges above or below 850 nm.

The light source elements 110 may comprise separate groups of light source elements 110 for each wavelength range and wherein each group is controlled by the wavelength selector depending on the desirous wavelength range to be emitted.

As alluded to above, the power button 130 may be cycled to operate the wavelength selector.

In alternative embodiments, the attachment 100 is configured to operably interface an electronic device (such as a mobile communication device) via a wireless interface, such as a Bluetooth interface to receive control instructions therefrom. In this regard, the electronic device may be operable to issue control instructions to the attachment 100 to control the wavelength selector.

The attachment 100 may further comprise an intensity selector control operably interfacing the light source elements 110. The intensity selector may be operable to cause the light source elements two emit light at at least two intensities.

For example, the intensity selector may be operable to cause the light source elements two emit light at intensities above or beneath 5 W/cm². The intensity selector may control the light source elements using pulse width modulation or alternatively control the number of light source elements 110 simultaneously operated to control the intensity.

Similarly, the intensity selector may be controlled by cycling the power button 130 or issuing control instructions from the electronic device wirelessly connected to the attachment 100.

In embodiments, the microcontroller may interface the light source elements and be programmed to calculate energy delivered measurements representing the total amount of energy delivered by the light source elements 110 during therapy sessions. The microcontroller may record the amount of energy delivered in Joules.

The microcontroller may calculate the energy delivered measurements according to at least one of operational intensity and operational duration of the light source elements 110. In further embodiments, the microcontroller may further calculate wavelength ranges associated with the energy delivered.

In further embodiments, the microcontroller may calculate muscle groups associated with the energy delivered. For example, the microcontroller may calculate that 500 J was delivered to the calf muscles whereas 250 J was delivered to the shoulder muscles. During therapy, the attachment 100 may be configured with an indication as to which muscle group is been targeted.

The attachment 100 may be configured to transmit the energy delivered measurements to electronic device wirelessly connected to the attachment 100.

In embodiments, the attachment 100 may comprise an indicator (such as an LED indicator, audible buzzer or the like) which is controlled by the microcontroller to indicate when the energy delivered measurements exceed a threshold. For example, the attachment 100 may be used to deliver an amount of energy to the calf muscles and wherein the microcontroller causes the indicator to indicate when the minimum amount of energy has been delivered.

In embodiments, the attachment 100 comprises an accelerometer operably interfacing the microcontroller. The microcontroller may be configured to take measurements from the accelerometer to detect when the impact surface 109 impacts the skin of the user during therapy. Furthermore, the microcontroller may be configured to control the light source elements during impact. For example, the microcontroller may extinguish or reduce the intensity of the light source elements 110 but may operate or increase the intensity of the light source elements 110 when detecting the impact surface 109 impacting the skin of the user.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. A red-light therapy massage gun attachment comprising a housing defining a massage gun driveshaft engagement at a proximal side of the housing and an impact surface at an opposite distal side of the housing and wherein the housing contains at least one light source element therein operative to emit light in a wavelength range between 600 nm and 1100 nm from the impact surface.

2. The attachment as claimed in claim 1, wherein the housing defines a disc-like head defining the engagement at a proximal side thereof and wherein a distal side of the disc-like head defines the impact surface and is convex.

3. The attachment as claimed in claim 2, wherein the radius of the impact surface is greater than the width thereof.

4. The attachment as claimed in claim 2, wherein the impact surface has a surface area greater than 3 cm².

5. The attachment as claimed in claim 1, wherein at least a portion of the impact surface is reflective.

6. The attachment as claimed in claim 1, wherein the distal side defines a recess therein and wherein the light source elements are located within the recess.

7. The attachment as claimed in claim 6, further comprising a transparent cover covering the recess.

8. The attachment as claimed in claim 7, wherein the transparent cover is shaped to continue a convex profile the impact surface.

9. The attachment as claimed in claim 8, wherein the transparent cover acts as a lens to at least one of focus or disperse light emitted by the light source elements.

10. The attachment as claimed in claim 6, wherein at least a portion of the distal side comprises reflective material.

11. The attachment as claimed in claim 1, wherein the distal side defines a recess therein and wherein the light source elements are located within the recess and wherein a surface of the distal side surrounding the recess is reflective.

12. The attachment as claimed in claim 11, wherein the surface of the distal side surrounding the recess comprises brushed aluminium.

13. The attachment as claimed in claim 1, wherein the distal side defines a recess therein and wherein the light source elements are located within the recess and wherein a surface of the recess is reflective.

14. The attachment as claimed in claim 13, further comprising a reflector located within the recess, the reflector defining shaped recesses for each light source element.

15. The attachment as claimed in claim 1, wherein the housing further comprises a battery therein for the light source elements.

16. The attachment as claimed in claim 15, wherein the housing comprises a recharging port operably coupled to the battery.

17. The attachment as claimed in claim 16, wherein the housing defines a disc-like head defining the engagement at a proximal side thereof and wherein a distal side of the disc-like head defines the impact surface and wherein the recharge port goes through the proximal side of the disc-like head.

18. The attachment as claimed in claim 15, wherein the engagement comprises electrical contacts to draw power from the massage gun in use for recharging the battery.

19. The attachment as claimed in claim 1, wherein the engagement comprises electrical contacts to draw power from the massage gun in use for the light source elements.

20. The attachment as claimed in claim 1, wherein the engagement comprises a shaft, thereby being configured for insertion within a barrel of the massage gun driveshaft.

21. The attachment as claimed in claim 20, wherein the shaft is sized to frictionally engage within the barrel in use.

22. The attachment as claimed in claim 20, wherein shaft comprises a proximal section and a distal section and wherein the proximal section is narrower than the distal section.

23. The attachment as claimed in claim 22, wherein a rubberised impact pad interfaces the sections.

24. The attachment as claimed in claim 20, wherein the shaft comprises an interference fit rim which fits within a corresponding recess of a massage gun driveshaft.

25. The attachment as claimed in claim 1, wherein the shaft defines a void therein and wherein the attachment further comprises a battery located within the void.

26. The attachment as claimed in claim 1, wherein the shaft exposes electrical contacts draw power from the massage gun in use for the light source elements.

27. The attachment as claimed in claim 1, wherein the engagement is recessed for insertion of a distal end of the massage gun driveshaft therein.

28. The attachment as claimed in claim 27, wherein the recess engagement exposes electrical contacts therein to draw power from the massage can in use for the light source elements.

29. The attachment as claimed in claim 1, wherein the light source elements provide a power of between 0.005 W/cm² and 10 W/cm².

30. The attachment as claimed in claim 1, further comprising a wavelength selector control operably interfacing the light source elements and wherein the wavelength selector is operable to cause the light source elements to emit light within at least two wavelength ranges.

31. The attachment as claimed in claim 30, further comprising a data interface configured to receive control signals from an electronic device and wherein the data interface operably controls the wavelength selector.

32. The attachment as claimed in claim 30, wherein the light source elements comprise a first group of light source elements configured to emit light at a first wavelength range and a second group of light source elements configured to emit light in a second wavelength range different from the first wavelength range and wherein the wavelength selector is operable to separately operate each group.

33. The attachment as claimed in claim 1, further comprise an intensity selector control operably interfacing the light source elements and wherein the intensity selector is operable to cause the light source elements to emit light at at least two intensities.

34. The attachment as claimed in claim 33, wherein the intensity selector control controls the intensity of the light source elements by pulse width modulation.

35. The attachment as claimed in claim 33, wherein the intensity selector control controls the intensity of the light source elements by controlling the number of light source elements operated simultaneously.

36. The attachment as claimed in claim 33, further comprising a data interface configured to receive control signals from an electronic device and wherein the data interface operably controls the intensity selector.

37. The attachment as claimed in claim 1, further comprising a controller interfacing the light source elements and wherein the controller is configured to calculate energy delivered measurements of energy delivered by the light source elements.

38. The attachment as claimed in claim 37, wherein the controller is configured to calculate the energy delivered measurements according to at least one of duration of operation and intensity.

39. The attachment as claimed in claim 37, wherein the controller is further configured to calculate the energy delivered measurements according to wavelength range.

40. The attachment as claimed in claim 37, wherein the controller is further configured to calculate the energy delivered measurements according to muscle group.

41. The attachment as claimed in claim 37, further comprising an indicator controlled by the controller to indicate when the energy delivered exceeds a threshold.

42. The attachment as claimed in claim 37, further comprising a data interface operably interfacing the controller and wherein the controller is operative to cause the data interface to transmit the energy delivered measurements via the data interface.

43. The attachment as claimed in claim 1, further comprising an accelerometer operably interfacing the light source elements and wherein the attachment is configured to detect impact using the accelerometer to operate the light source elements during impact.
